(19) United States
(12) Patent Application Publication
(10) Pub. No.: US 2012/0077219 A1
(43) Pub. Date: Mar. 29, 2012

(54) VIBRATING MICROPLATE BIOSENSING FOR CHARACTERISING PROPERTIES OR BEHAVIOUR OF BIOLOGICAL CELLS

(76) Inventors: Xianhong Ma, Sambourne (GB); Zhangming Wu, Chuzhou City (CN); Peter Nigel Brett, Sambourne (GB); Michael T. Wright, Stratford upon Avon (GB); Helen R. Griffiths, Solihull (GB)

(21) Appl. No.: 13/375,206

(22) PCT Filed: Jun. 25, 2010
(86) PCT No.: PCT/GB2010/001252

§ 371 Date: Nov. 29, 2011

(30) Foreign Application Priority Data
Jun. 30, 2009 (GB) 0911331.7

(51) Int. Cl. C12Q 1/02; C12M 1/34

(52) U.S. Cl. 435/29; 435/288.3

(57) ABSTRACT

There is described a method of characterising a property or behaviour of at least one biological cell. The method comprises the steps of: providing a microplate; submerging at least one surface of the microplate in a cell culture medium such that at least one biological cell to be characterised is in contact with the microplate; vibrating the microplate; providing a plurality of mutually spaced sensors coupled to the microplate; obtaining a respective sensory data time series from each sensor during vibration of the microplate, the microplate and the sensors being arranged such that the obtained sensory data time series are not independent from one another; and processing the sensory data time series so as to characterise a property or behaviour of the at least one biological cell. A corresponding system for characterising a property or behaviour of at least one biological cell is also described.

### FIELD OF THE INVENTION

The present invention relates to a method of and system for characterising a property or behaviour of at least one biological cell. The method and system may be used, for example, to characterise cell properties and behaviour such as cell propagation, cell polarity, cell movement, cell growth, cell contraction, cell migration, cell proliferation, cell differentiation, and microbe growth in vitro.

### BACKGROUND OF THE INVENTION

Currently measurements of physical properties and behaviour of biological cells are mainly performed under a microscope using microscopy imaging systems. The tasks of cell culture, monitoring and manipulation can be tedious and time consuming. Cell responses to external stimuli are frequently difficult to visualise in real time.

The use of mechanical transducer principles to design sensors for MicroElectroMechanical Systems (MEMS) is of growing interest for engineers, physicists, chemists and biologists. The most widely applied mechanism is the microcantilever. It has been used in MEMS to build sensors of different kinds, such as force sensors with integrated tips for AFM, bimetallic temperature and heat sensor, mass loading sensor, medium viscoelasticity sensor, and thermogravimetric sensor and stress sensor. The merging of micro-fabrication techniques, surface functionalization biochemistry and cantilever sensing methods offers opportunities to develop bio-sensors for clinical and environmental purposes. The article “A high-sensitivity micromachined biosensor” by Basel et al. (Biosensors and Bioelectronics, Volume 12, Issue 8, 1997, Page iv) proposes to detect the presence of receptor-coated magnetic beads that stick to the functionalised surface using microcantilevers. The article “Translating biomolecular recognition into nanomechanics” by Fritz et al. (Science, Volume 288, Issue 5464, Apr. 14, 2000, Pages 316-318) monitors ssDNA hybridisation with two microcantilevers parallel where their differential deflections allow discrimination of two identical 12mer oligonucleotides with a single base mismatch.

Nonetheless, there is a need for a micro sensing system able to achieve dynamic and contact measurement of basic biological processes such as cell movement, contraction, migration, proliferation or differentiation and microbe growth in vitro. Many applicable areas of such sensors have been proposed. The article “Engineering cellular microenvironments to improve cell-based drug testing” by Bhadriraju and Chen (DDT, Volume 7, Issue 11, Pages 612-620, June 2000) suggests using engineering cellular microenvironments to improve cell-based drug testing. The article “Morphological changes and cellular dynamics of oligodendrocyte lineage cells in the developing vertebrate central nervous system” by Ono et al. (Developmental neuroscience, Volume 23, Issue 4-5, Pages 346-355, 2001) suggests that the study of morphological changes of oligodendrocyte lineage cells and their cellular dynamics including cell motility and proliferation will provide insight of the potential molecular mechanisms of OPC dispersal throughout the central nervous system. The article “The Effect of Cell Division on the Cellular Dynamics of Microinjected DNA and Dextran” by Ludtke et al. (Volume 5: 579-588 (2002), Molecular Therapy, 6(1), July 2002, Page 134) shows the effect of cell division on the cellular dynamics by microinjecting DNA and Dextran.

In order to provide another dimension in cell measurement other than the microscope, the present invention seeks to provide a micro sensing method and system for improved detection and monitoring of cell growth and dynamical properties such as movement, contraction, morphology change, migration in vitro. The method and system described herein are intended to complement presently available imaging systems.

### SUMMARY OF THE INVENTION

According to a first aspect of the present invention, there is provided a method of characterising a property or behaviour of at least one biological cell. The method comprises the steps of: providing a microplate; submerging at least one surface of the microplate in a cell culture medium such that at least one cell to be characterised is in contact with the microplate; vibrating the microplate; providing a plurality of mutually spaced sensors coupled to the microplate; obtaining a respective sensory data time series from each sensor during vibration of the microplate, the microplate and the sensors being arranged such that the obtained sensory data time series are not independent from one another; processing the sensory data time series so as to characterise a property or behaviour of the at least one biological cell.

Thus, to overcome the difficulties associated with measuring the physical properties and behaviour of biological cells using microscopy imaging systems, and to enable consistent quantitative measurement of cell properties and behaviour, the present invention provides an integrated cell monitoring method by using the information derived from the dynamics of a plate submerged in cell culture fluid and advanced system identification techniques. The present invention overcomes the frequent difficulties associated with visualising cell responses to external stimuli in real time using microscopy imaging systems. The integration of the plate dynamics and automated time series analysis can provide a history of cell dynamical information without relying totally on continuous image monitoring. No existing technologies that can provide the cell information that the present invention is able to provide. Furthermore, the present invention provides a natural cell grow environment, with no florescence or laser bleaching, for example. The present invention also enables real-time continuous monitoring of biological cells. The present invention has high sensitivity and a fast response time.

In addition, the dynamics of microplates are more complex than the dynamics of the well known microcantilevers discussed above. Due to their more interesting dynamical characteristics, microplates can provide additional information as a micro sensing medium as compared to microcantilevers. Also, microplates offer new benefits for maintaining the natural culture environment of cells (and microbes) due to the fact that viable cells can be maintained on their surfaces in the cell culture medium.

In one embodiment of the invention, the processing step of the cell characterisation method comprises: specifying a cell dynamic behaviour category; and processing the sensory data time series so as to determine whether the dynamic behaviour of the at least one cell is in the specified cell dynamic behaviour category. In another embodiment, the processing step comprises: specifying a cell property; and processing the sensory data time series so as to determine a measurement of the specified property of the at least one cell.

The processing step may comprise analysing the sensory data time series in one or more of the time domain, the frequency domain and the wavelet domain. The processing step may comprise analysing frequency response functions (FRFs). The processing step may comprise using a neural network and/or Karhunen-Loeve decomposition.

In one embodiment, the microplate is vibrated periodically. In another embodiment, the microplate is vibrated randomly.

According to a second aspect of the present invention, there is provided a system for characterising a property or behaviour of at least one biological cell. The system comprises: a container for cell culture medium; a microplate disposed within the container such that, when the container is at least partially filled with cell culture medium, at least one surface of the microplate is submerged in the cell culture medium; at least one actuator operable to vibrate the microplate; a plurality of mutually spaced sensors coupled to the microplate, each sensor being operable to provide a respective sensory data time series during vibration of the microplate, the microplate and the sensors being arranged such that the provided sensory data time series are not independent from one another; and a processor operable to receive the sensory data time series from the sensors and to process the received sensory data time series so as to characterise a property or behaviour of at least one biological cell in contact with the microplate.

The microplate boundary conditions may be selected from clamped, cantilever, free and point-supported, for example.

The sensors may be selected from piezoresistive gauge sensors, optical sensors, strain sensors and acceleration sensors.

In one embodiment, the at least one actuator comprises a piezoelectric transducer. In another embodiment, the at least one actuator comprises a sonic actuator.

In one embodiment, the container is a Petri-dish.

Other preferred features of the present invention are set out in the appended claims.

### BRIEF DESCRIPTION OF THE DRAWINGS

Embodiments of the present invention will now be described by way of example with reference to the accompanying drawings in which:

FIG. 1a is a schematic plan view of a biosensing platform for a biosensing system in accordance with the present invention;

FIG. 1b is a schematic side view of the biosensing platform of FIG. 1a;

FIG. 2 is a schematic perspective view of a biosensing platform for a biosensing system in accordance with the present invention; and

FIG. 3 is a schematic representation of the set-up of the automatic biosensing system showing the principal functions and elements which are used to build the nonlinear processing model.

FIG. 4 is a Scanning Electron Microscope (SEM) image of an integrated biosensing platform.

FIG. 5 shows a Laser Scanning Micrometer (LSM) image of endothelial cells coated on the surface of a microplate of a biosensing platform.

FIG. 6 illustrates a dynamic testing device for a biosensing platform.

FIGS. 7, 8 and 9 illustrate the frequency response functions (FRFs) of three different types of microplates under three different cell densities.

FIG. 7 uses a 100 μm square C-F-F-F microplate;

FIG. 8 uses a 200 μm square C-F-C-F microplate; and

FIG. 8 uses a 300 μm square C-C-C-C microplate. In each case, (a) and (b) show endothelial cells coating on the surface of the microplate, and (c) shows normalised velocity amplitude according to cell density.

FIGS. 10, 11 and 12 illustrate the trends of FDRₙ as the amount of cells is increased for three tested microplates (No. I, No. II and No. III respectively). FDRₙ is a Frequency Difference Ratio evaluated as the normalized resonant frequency difference between the cell-loaded and cell-free membrane at a measured resonance mode n.

FIG. 13 shows the AFDR index of the three micro-membranes of FIGS. 10, 11 and 12 in each batch of experiments. AFDR is the average of all measured FDRₙ.

FIG. 14 illustrates the quantification of cell population based on a simple image processing technique.

FIG. 15 is a schematic diagram of a BP neural network used for cell identification.

FIG. 16 shows predicted results on the CDR of sample numbers 15 to 18 using the BP neural network trained using samples 1 to 14.

### DETAILED DESCRIPTION OF A PREFERRED EMBODIMENT

A micro/nano-scale biosensing system in accordance with the present invention includes a container (not shown) for cell culture medium (e.g. cell culture fluid). A biosensing platform is disposed within the cell culture medium container.

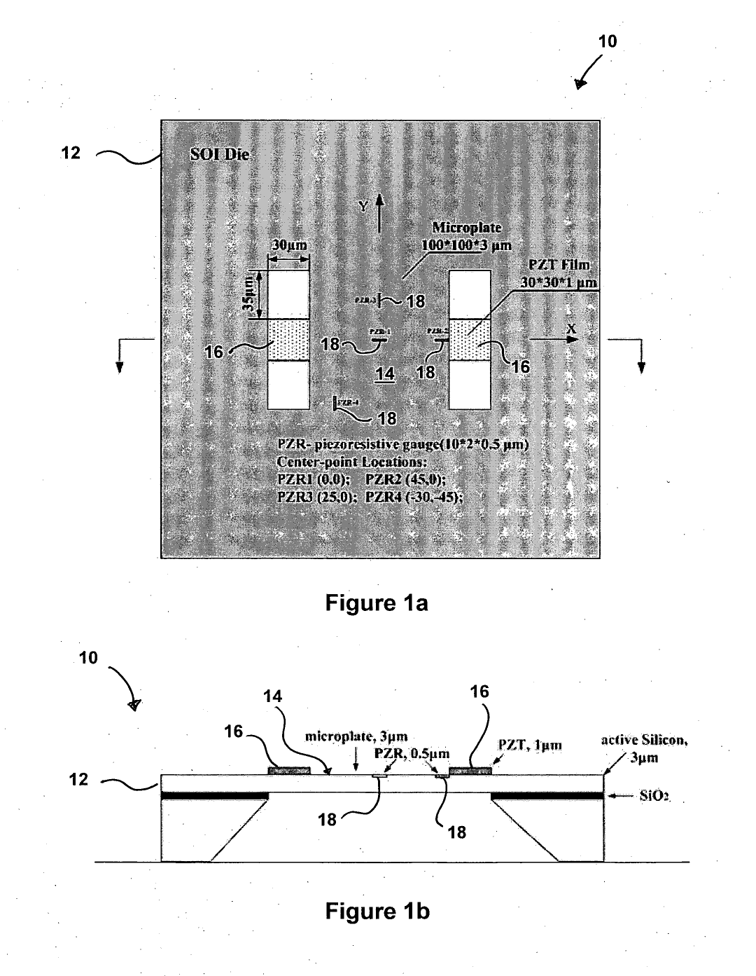

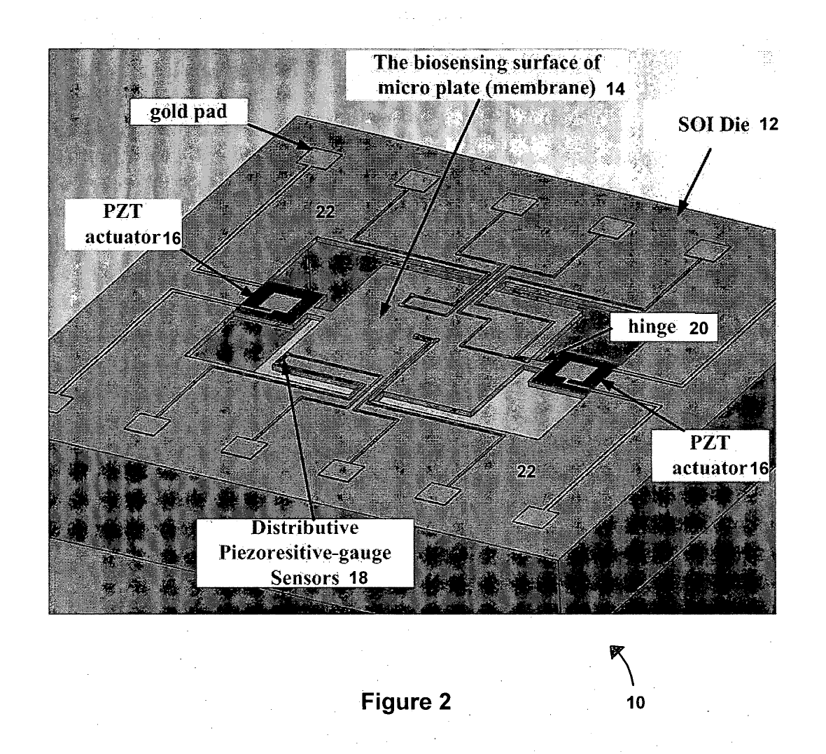

FIGS. 1a and 1b show a plan view and a side view of one embodiment of the biosensing platform 10. A slightly different embodiment is shown in perspective view in FIG. 2.

The biosensing platform 10 is largely formed from an SIO substrate 12. The biosensing platform 10 includes a microplate 14, two actuators in the form of piezoelectric transducers (PZTs) 16, four mutually spaced sensors 18, and a power input (not shown). The biosensing system further includes a processor (not shown) which may form part of the biosensing system. The biosensing platform 10 is designed to be able to work in fluid (e.g. water), with a good bio-sensitivity under the high damping conditions. The biosensing platform 10 may be either single-sidely or double-sidely immersed in cell culture fluid within the fluid container to maintain the natural cell living environment. The biosensing platform 12 uses materials that are biocompatible, such as silicon and gold, such that biological cells can use it as a natural growth ground when it is submerged in cell culture medium.

The microplate 14 is a thin micromachined membrane which acts as a micro/nano-scale sensing platform. Micromachined membranes (plate/diaphragm) are a promising mass sensing structure to replace the microcantilever. Compared with microcantilevers, micro-membranes potentially have larger sensing area, higher sensitivity in liquid and less fragility. Moreover, micro-membranes have the same advantages as the microcantilever in the application of mass sensing. The microplate 14 is deformable. The microplate 14 has dimensions in the range of tens to hundreds of microns in the X- and Y-directions. For example, the microplate 14 may have dimensions from tens to thousands of microns (e.g. 100-400 μm) in the X- and Y-directions. The depth of the plate in the Z-direction is about 3 μm as shown in FIG. 1b, but a depth in the range of a few nanometers up to tens of microns would also be appropriate. These dimensions are intended to be representative rather than limiting. The microplate 14 may be supported by means of a variety of different boundary conditions (e.g. clamped, cantilever, free and point supported, etc.). In the embodiment of FIG. 2, the microplate 14 is rectangular. The microplate 14 is supported by four hinges 20, each hinge being located centrally along a respective one of the four sides of the microplate 14. This is one example of the microplate boundary conditions.

Actuators (i.e. excitation sources) are used to vibrate the microplate 14 within the cell culture medium. In the embodiment of FIGS. 1a and 1b, the actuators are two PZT (Lead Zirconate Titanate) thin films 16. The PZTs 16 are deposited inside or beside the region of the microplate 14 to provide powerful excitation force with limited energy consumption. Thus, the biosensing system is designed to be capable of self-excitation. As an alternative to the use of PZT actuators, the microplate 14 could instead be actuated by sound excitation. The actuators 16 may be integrated into the biosensing platform 10. The actuators 16 may be integrated into the microplate 14.

The biosensing system is designed to be capable of self-sensing. Four distributive Piezoresistive-gauge (PZR) sensors 18 are shown in FIGS. 1a and 2. The sensors 18 are placed at well-selected locations for obtaining the whole-domain dynamical/vibrational information of the microplate 14. The sensors 18 may be embedded in the microplate 14. Advanced micro-fabrication techniques are used to produce the sensing elements 14 and associated connecting tracks 22 shown in FIG. 2. As an alternative to the use of PZR sensors, different sensor types may be used, e.g. optical, strain, or acceleration sensors. The sensors 18 may be integrated into the biosensing platform 10. The sensors 18 may be integrated into the microplate 14. The positions of the sensors 18 can be optimised with regard to maximising sensitivity for discrimination and maximising the range of high performance over the microplate surface.

The PZT actuators 16 and piezoresistive-gauge sensors 18 are of good compatibility with CMOS circuits and are easily integrated with other electronic components. The electronic parts of the biosensing platform 10 (e.g. the electrode wires, gold pads, and connecting probes) are sealed with biocompatible material. The whole biosensing platform 10 is packaged by using standard DIL (Dual in-line). The signal flow (input and output signals) may be processed either through external processing instruments or internal electronic chips.

Advanced tools and processes are used for the micro/nano fabrication of the biosensing platform, including optical and electron beam lithography, plasma etching and a focused ion beam tool capable of etch and deposition for rapid prototyping in nanofabrication.

In use, the biosensing system is used to discriminate a single cell or a collected group of cells' property or behaviour.

The cell culture medium container is partially or completely filled with cell culture medium. The microplate 14 of the biosensing platform 10 is placed into the cell culture medium container such that at least one surface of the microplate 14 is submerged or immersed in the cell culture medium. For example, the microplate 14 may be completely submerged within the cell culture medium. Alternatively, only the bottom surface of the microplate 14 may be submerged within the cell culture medium. The submersion of the microplate 14 within the cell culture medium enables biological cells within the cell culture medium to use the microplate 14 as a natural growth ground. Thus, there is in contact with the microplate 14 at least one biological cell whose property/behaviour is to be characterised by the biosensing system and method.

The microplate 14 is then vibrated by the actuators (e.g. PZTs 16). The microplate 14 can be excited periodically (e.g. using a sinusoidal function) or randomly with a wide frequency band random signal (e.g. Pseudo Random Binary Signals, white noise, or burst random, etc.). The type of excitation/vibration will vary depending on the implementation purposes. The microplate 14 is vibrated because the contacting biological cells do not impose a significant force on the microplate 14 on their own. The contacting biological cells impact on the mass, stiffness and strain properties of the microplate 14. Thus, measurements of these variables (e.g. strain gauge measurements) may be used to quantify the effect of the contacting biological cells on the microplate 14 and to thereby infer the properties/behaviour of the cells to be characterised. Using a static microplate 14, the deflection of the microplate 14 by the contacting cells is very small which makes it difficult to detect the signals in, for example, the field of strain in the microplate 14. Consequently, it can be difficult to infer the properties/behaviour of the cells to be characterised. Thus, the microplate 14 is advantageously vibrated towards and away from the contacting biological cells so as to produce a stronger signal in the field of strain in the microplate 14 due to the presence of the cells. Alternatively/additionally, the microplate 14 could be vibrated in other directions rather than solely towards and away from the contacting biological cells. Vibrating the microplate 14 has other advantages too: the vibrations provide additional information about the dynamical character of the microplate 14 (e.g. natural frequency shifts, mode shape changes, and other nonlinear coupling effects). This additional dynamical information may also be used to characterise properties or behaviour of the contacting cells.

Whilst the microplate is being vibrated, respective sensory data time series are obtained from each sensor 18. The microplate 14 is a continuous medium that provides a nonlinear coupling between the contacting biological cells and the sensors 18. Thus, the sensors 18 are coupled through the deformation response of the microplate 14 to biological cells in contact with the microplate 14 such that the sensory data time series are not independent from one another. This means that, although the sensors 18 receive local sensory data from the microplate 14, the sensory data time series from a particular sensor 18 may show cell movement remote from that sensor 18. In other words, the sensors 18 indirectly sense properties/behaviour of the biological cells via the microplate 14. The biosensing platform uses the variation of its dynamic/vibrational characteristics as the information source to sense the surface-contact biological cells and particles. By interpreting the simultaneous collective sensed responses of the sensors 18, the nature of any cell disturbance can be discriminated in such a way as to determine a property or behaviour of the contacting biological cells. The sensors 18 respond in a non-independent (i.e. coupled) manner due to the presence of the microplate 14, which acts as the coupling mechanism between the sensors 18. Due to the coupled nature of the system, only a relatively small number of discrete sensors 18 are needed on the microplate 14. The resolution of the biosensing platform 10 is not limited to the pitch separating the sensors 18 and can therefore be used to detect variations much smaller than the smallest scale of manufacturing. Furthermore, due to the coupled nature of the system, the sensors 18 may be provided on a surface of the microplate 14 other than the cell-contacting surface. This adds to the robustness of the approach.

Having obtained the sensory data time series, these time series are processed using advanced system identification methodologies and embedded IT tools so as to characterise a property or behaviour of the at least one biological cell. During the processing step, the sensory data time series from each sensor is processed together with the sensory data time series from each of the other sensors (i.e. the data is processed collectively). The processing is nonlinear. For example, nonlinear signal processing techniques such as neural networks or Karhunen-Loeve decomposition may be used to process the coupled simultaneous time series, either in time or frequency domain.

The nonlinear processing model utilises the dynamical information in the sensory data time series to detect cell properties and behaviour. Spatial dynamical information from the microplate 14 (e.g. mode shapes, coupling between the sensors, etc.) is used to derive spatial dynamical information regarding the cells on the microplate 14 (e.g. polarity, stem cell growth). System identification tools are used to correlate the output of the processing step with the property or behaviour of the cell/cells/tissues which it is desired to characterise. In other words, the dynamical information will be correlated to the state and characteristics of the dynamical cell properties. For example, the property or behaviour of interest may be one that is essential in drug development, microbiological and tumour screening, or stem cell biology. This can include static or dynamic properties or behaviours, such as propagation, polarity, cell movement/growth, contraction, migration, proliferation or differentiation and microbe growth in vitro. The present system and method may be deployed to derive size, shape and movement information on the contact of a cell or cells during the processes of cell culture, cell manipulation and cell surgery. One aim of the biosensing method and system is to detect the change in cell morphology, migration, proliferation, differentiation, and contractility during cell culture and growth processes. The dynamic characteristics of the microplate 14 (such as velocities and accelerations) are used to infer the information required using the relatively few sensing elements 18 through system identification algorithms. The dynamic response signals of the microplate 14 (i.e. the sensed data time series) are applied to intelligent time series identification algorithms to derive the desired cell property or behaviour information. Discrimination of the cell properties/behaviour is achieved by using embedded information tools. Outputs can be in discrete form or continuous with a variety of descriptors according to the aims of the application.

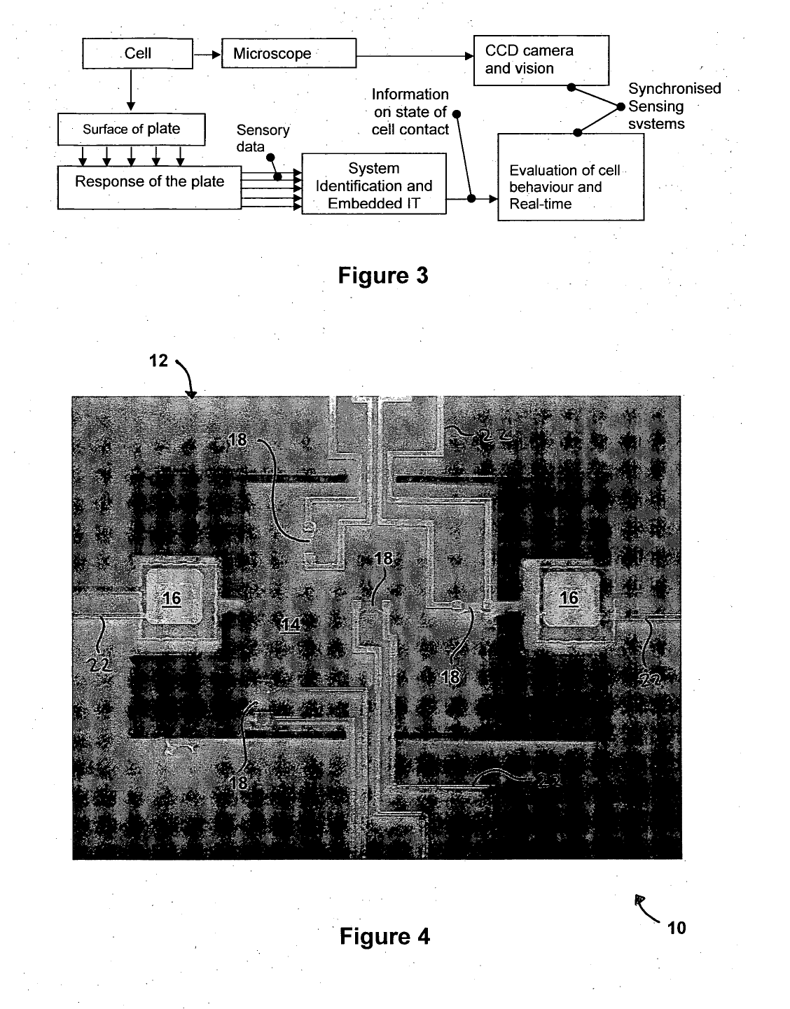

The nonlinear processing model used in the biosensing system is trained using training data. The microplate 14 vibrates differently under different loading conditions. Therefore, the nonlinear processing model takes into account the known dynamics of the microplate 14 in a liquid environment. For example, the microplate dynamics will be affected by the acoustic pressure waves caused by the interaction of the microplate 14 with the cell culture medium (which generally has a slightly higher density than water). Thus, the nonlinear processing model is built with results from using a micro scanning laser vibrometer to investigate the micro scaling effects on the dynamics and sound radiation of the microplate 14 in fluid. The micro scanning laser vibrometer is used to measure the dynamics and sound radiation of the microplate 14 in liquid, such as natural frequencies, natural modes, forced response at certain forcing conditions. Thus, use of the micro scanning laser vibrometer enables an appropriate nonlinear processing model (e.g. neural network) to be created. In other words, results obtained using the micro scanning laser vibrometer are used as training data for the neural network, for example. Through the simulation of dynamics of a submerged microplate, the nonlinear processing model can be set up to infer the loading conditions from the sensory data time series of the vibrating microplate 14. In the modelling process, the displacements/velocities/accelerations at different sensing positions can be obtained through simulation. The nonlinear processing model that relates the parameters extracted from the dynamic signals (i.e. the sensory data time series) to the external forces/loadings is set up using system identification techniques such as Karhunen-Loeve decomposition, wavelet analysis and artificial neural network methods. The nonlinear processing model can be tested and validated by experiments using the Pseudo-Random Binary Sequence (PRBS) excitation and identification method. The advantage of PRBS signals is that they possess the property where their autocorrelation function is a close approximation to the impulse function. The dynamics of all frequencies are excited by PRBS signals. Thus dynamics of the microplate 14 under any forcing conditions can be derived. The validated model can then be used to deduce the force/loading applied on the microplate 14 by the sensory data time series of the vibration at different locations on the microplate 14. When the system identification technique is applied to cell/tissue monitoring, the state and condition of cell dynamics is deduced. The acceleration amplitude of the microplate 14 is less when the microplate 14 is submerged, this is due to the fact that each mode generates an acoustic pressure in the plane of the microplate 14, the normal modes become coupled in liquid. Still, by placing the sensors 18 in appropriate positions, the principal mode shapes can be related to the loading and dynamic conditions on the microplate 14 through proper system identification techniques. The correlation of cell behaviour to the transients detected and information derived from the microplate sensing surface is taken into account in the nonlinear processing model. A CCD camera deployed through a microscope system is used to monitor visible behaviour and the output is correlated through a synchronised vision processing system with the information and sensory data outputs from the biosensing system. The functions of the experimental set-up are shown schematically in FIG. 3.

As mentioned above, biological cells exhibit a range of responses due to external stimuli that are frequently difficult to visualise in real time using existing microscopy imaging systems. The biosensing method and system described herein enhance the measurement available from microscopy imaging systems and significantly add to the level of information available to cell biologists. Three possible applications of the present microplate dynamics method and system are described below. In each of the application examples below, the biosensing platform is submerged in the culture medium. Cells then attach to the microplate and grow on it.

The first potential application relates to membrane polarity. Leukocytes such as monocytes and neutrophils do not show any polarity at rest, however, in response to chemotactic stimuli, membrane receptors become polarised and move toward the direction of stimulus. Similarly, tumour metastic potential can be defined as ability to polarise and colonise new sites. Current techniques for analysing cellular migration are based on movement through porous membranes in response to trigger, where the lack of sensitivity of the procedure predicates large sample size in order to visualise migration. In analysing the responses of tumours to metastatic inhibitors, only small sample sizes are available, and there is a need to improve sensitivity of analysis. Using the system and method of the present invention, the redistribution of cell membranes and migration across the microplate can be examined as an indicative marker of the responsiveness of neutrophils to a range of chemotactic agents and of tumours to matrix metalloproteinase inhibitors (e.g. TAPI) which may inhibit metastatic potential. The development of such a technique offers much enhanced sensitivity and speed in drug development.

The second potential application relates to cell proliferation, differentiation and apoptosis. During embryogenesis and within actively regenerating tissue such as tumours, resident cells continue to divide. Cell proliferation is visualised as an increase in cell number, where thousands of cells may be studied at any one time. Again, such a technique is crude and requires large cell numbers, where inevitably there will be a mixture of cells under study. There is a need for a simple technique which will allow sensitive determination of cell growth using small sample sizes. To achieve this, microdissection may be used to extract single cells from tumours, and the rate of cell division will be determined as a change in size and mass using microplate dynamics in accordance with the method and system of the present invention. Responsiveness over varying times and dose ranges to a range of chemotherapeutic agents, including methotrexate, can be studied as the change in cell shape induced during differentiation or apoptosis.

The third potential application relates to muscle cell development from stem cells. The lack of progenitor definition of stem cells allows their development into a range of mature cells types. Stem cell biology thus contributes to the rapidly growing area of stem cell bioengineering; the manipulation of environmental signals influencing cell survival, proliferation, self-renewal and differentiation. In this way multivariate analytical approaches have been used with success to optimise different stem cell culture processes. Again this process may be enhanced through the use of technologies which sense small changes in morphology and function, including those with a contractile phenotype. The maturation of stems cells into smooth muscle cells may be induced, and the efficiency of maturation into contracting muscles can then be analysed on a single cell basis using microplate dynamics in accordance with the method and system of the present invention.

### Experimental Results

Further details are now provided regarding experiments which have been performed using biosensing platforms 10 in accordance with the present invention. In particular, a biosensing system based on a micromachined rectangular silicon membrane (i.e. the microplate 14) has been investigated. A distributive sensing scheme monitors the dynamics of the sensing structure. An artificial neural network algorithm is used to process the measured data and to identify cell presence and density. Thus, in these experiments, the cell properties to be characterised are cell presence and density. Without specifying any particular bio-application, the investigation was mainly concentrated on the performance testing of this kind of biosensor as a general biosensing platform. The biosensing experiments on the microfabricated membranes involve seeding different cell densities onto the sensing surface of membrane, and measuring the corresponding dynamics information of each tested silicon membrane in the forms of a series of frequency response functions (FRFs). All experiments were carried in a cell culture medium to simulate a practical working environment. The EA.hy 926 endothelial cell lines were chosen for the bio-experiments. The EA.hy 926 endothelial cell lines represent a particular class of biological particles that have irregular shapes, non-uniform density and uncertain growth behaviours, which are difficult to sense using traditional biosensors. The final predicted results reveal that the methodology of a neural-network based algorithm to perform the features identification of cells from distributive sensory measurement, have great potential in the applications of biosensors.

It should be noted that these experiments are presented by means of example only, and no aspect thereof should be considered as limiting to the scope of the present invention as set out in the appended claims.

### 1. Fabrication of Membrane Biosensinq Devices

In these experiments, the silicon membrane (i.e. the microplate 14) was fabricated using the standard micromachining techniques from silicon on insulator (SOI) wafers. The membrane was created by inductively coupled plasma (ICP) using the Deep Reactive Ion etching (DRIE) process from the back side of the SOI wafer (i.e. the SOI substrate 12), stopping at the buried oxide layer. Boundary conditions of the membrane were also defined by DRIE from the top side of the wafer, using the buried oxide as stop layer. The buried oxide layer was finally removed to form the boundary holes. Three different boundary conditions of the micro-membranes were fabricated and tested: two opposite edges clamped and the other two edges free (C-F-C-F), cantilever (C-F-F-F) and all edges clamped (C-C-C-C). All of the membranes are designed to be square and with lengths 100 μm, 200 μm or 300 μm.

To dynamic test the above membrane structure, an external actuator was used for excitation and a laser vibrometer was used for vibration measurement. A large number of biological experiments were implemented on those membranes to examine their biosensing performances.

FIG. 4 is a Scanning Electron Microscope (SEM) image of an integrated microsystem (i.e. the biosensing platform 10) based on a 100 μm square sensing membrane, which was manufactured with distributive piezoresistive sensors (i.e. the sensors 18) and PZT actuators (i.e. the actuators 16). Such a microsystem enables the device to be capable of self-sensing and self-excitation. This microsystem can be embedded into an electronic circuit to build a lab-on-chip system.

For the fabrication of distributive piezoresistive sensors, a 500 nm-thick poly-silicon layer was deposited onto the oxidised device layer of a SOI wafer by low pressure chemical vapour deposition (PCVD). This layer was then doped by ion beam implantation using a 50 Kev Boron source giving a doping density of 1e15 to enhance the piezoresistive deflection sensitivity. The two sensor shapes were formed by photo-lithography and subsequent reactive ion etching (RIE).

In the PZT film fabrication, a sandwiched structure of a 100 nm-thick Pt/Ti bottom electrode, a 1 μm PZT film and a 100 nm-thick Pt top electrode was deposited on the SOI. The top and bottom electrodes were deposited by evaporation using e-beam evaporator systems, the deposited PZT was deposited as a spin on sol-gel which is then annealed to produce the required PZT film. The top and bottom electrodes are patterned and etched by ion beam milling. The redundant PZT material was wet etched.

### 2. Biological Experiments

The human hybrid EA.hy 926 cell used in these experiments is derived from the fusion of the human umbilical vein endothelial cells with A549/8 human lung carcinoma cell line. EA.hy 926 is a permanent human endothelial cell line that expresses highly differentiated functions characteristic of human vascular endothelium. Human EA.hy 926 endothelial cell lines are maintained in 30 ml Dulbecco's Modified Eagle's Medium (DMEM), supplemented with 10% FBS, streptomycin 100 μg/ml and penicillin 100 U/ml, and 10 ml HAT (100 μM hypoxanthine, 0.4 μM aminopterin, 16 μM thymidine). Cells were cultured in an incubator at 37° C. with an atmosphere of 5% CO₂ and 95% air. Cells were grown in a 75 cm² flask and passaged when reaching ˜90% confluence. Once cells roughly reached 90% confluence the media was removed and the cells washed with 5 ml phosphate buffered saline (PBS). The process of passage of EA.hy 926 cells is that briefly cell culture media was removed from the cells and cells were then washed with 10 ml sterile PBS until the media appeared without colour. EA.hy 926 cells were then detached by the addition of 2.5 ml trypsin with a 3 minute standard incubation. Cell clusters were also dispersed for uniform distribution by repeated pipetting with 5 ml new DMEM media.

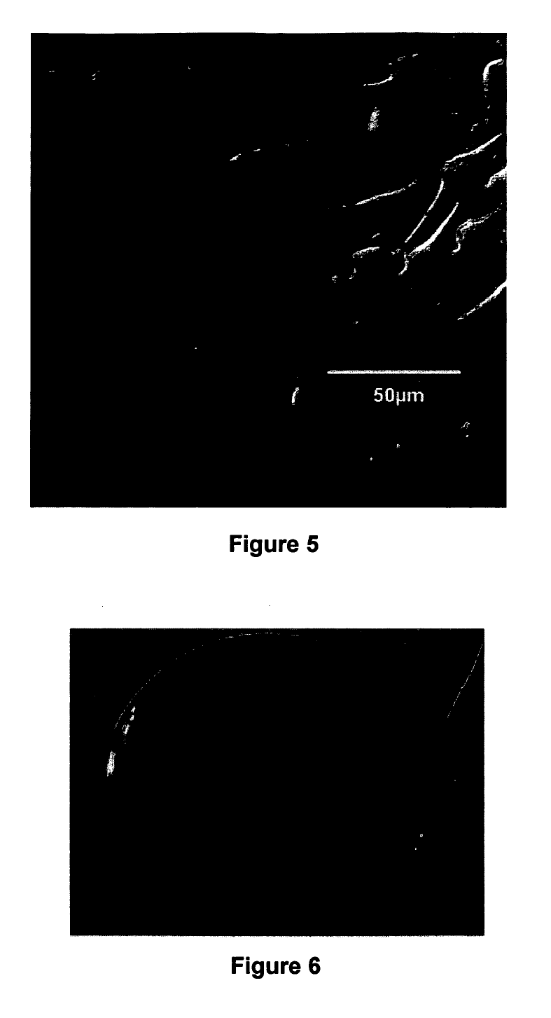

FIG. 5 shows a Laser Scanning Micrometer (LSM) image of endothelial cells coated on the surface of a micro-membrane. The endothelial cells are tightly adherent to the silicon surface showing a typical spreading pattern.

The biological experiment is separated into two phases: (1) seeding a certain amount of cells on the membrane, and (2) measuring the corresponding dynamics of the membrane. The dynamic testing device is illustrated in FIG. 6. Identical micromembranes were repeatedly used several times for obtaining a batch of experimental results with different densities of cells. Each experiment was performed as follows:
1. Initially, silicon micromembranes were cleaned and sterilised using washes (ethanol and acetone mixture), autoclaving and UV light irradiation.
2. Before seeding cells on the micro-membranes, the cell density of suspension during the process of passage was established. The numbers of viable cells was estimated by taking 20 μl of the cell suspension and mixing it with a 20 μl trypan blue. A cell count was then performed for this new mixture by using improved Neubauer haemocytometer. Once the cell density was established, a 5 ml cell suspension of EA.hy 926 cells of known density was made up using the media. By controlling the incubation time, various cell density and distribution on the membrane surface can then be achieved.
3. Cell distribution on the membrane sensing surface was recorded using a LSM (laser scan microscopy) image. The density or distribution of cells can be quantified based on this LSM image.
4. The dynamics of membranes with adherent cells were measured through the base-excitation apparatus of FIG. 6. The FRF data for each specific micro-membrane with cells and without cells were compared to infer the information of cells, which was recorded in the LSM scanned images.
5. Finally, the cells were removed from the surface of micro-membranes, and after repeating step 1 cleaning process, the re-sterilised micromembrane was used for the next experiment.

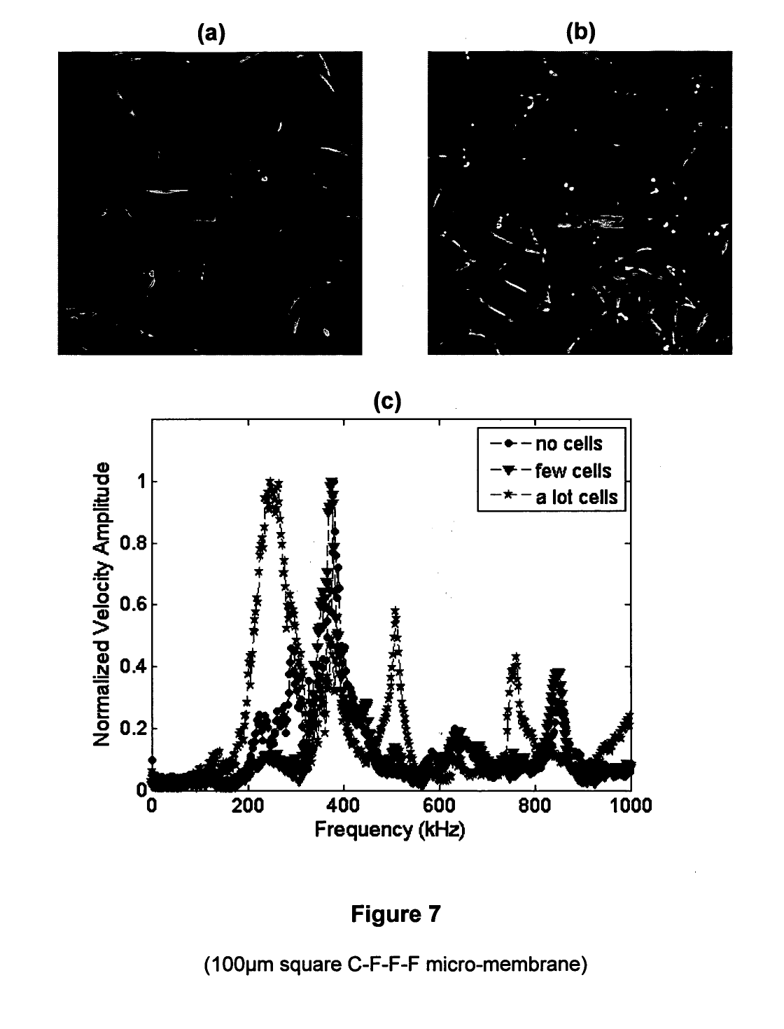

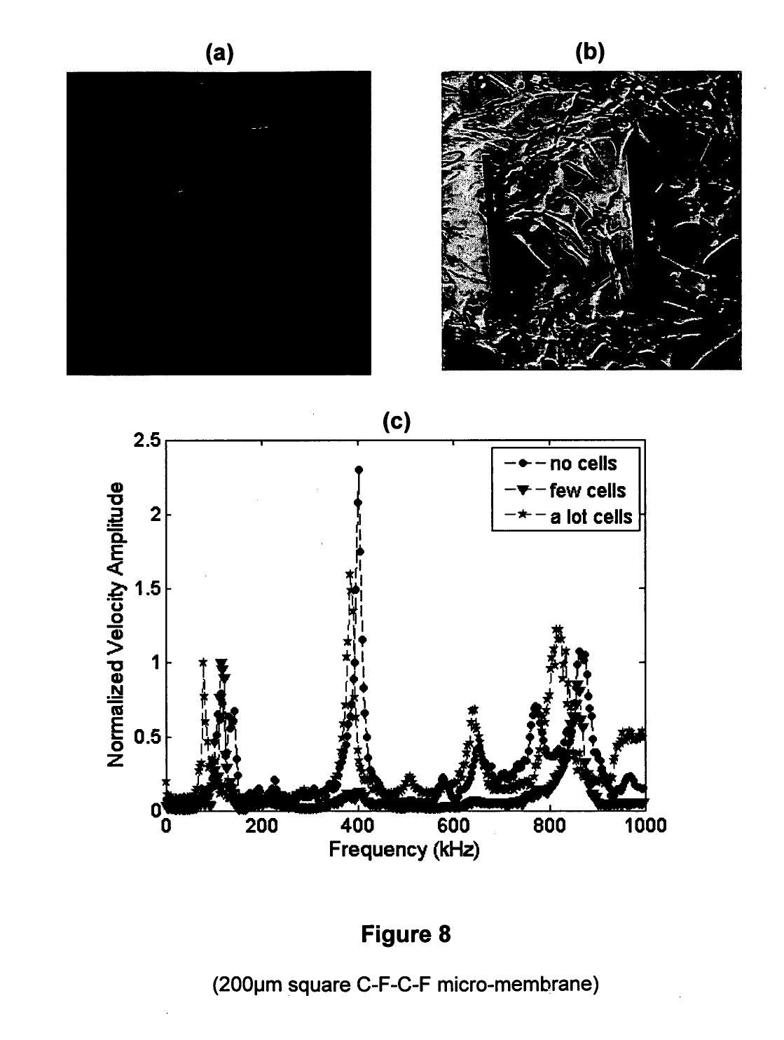

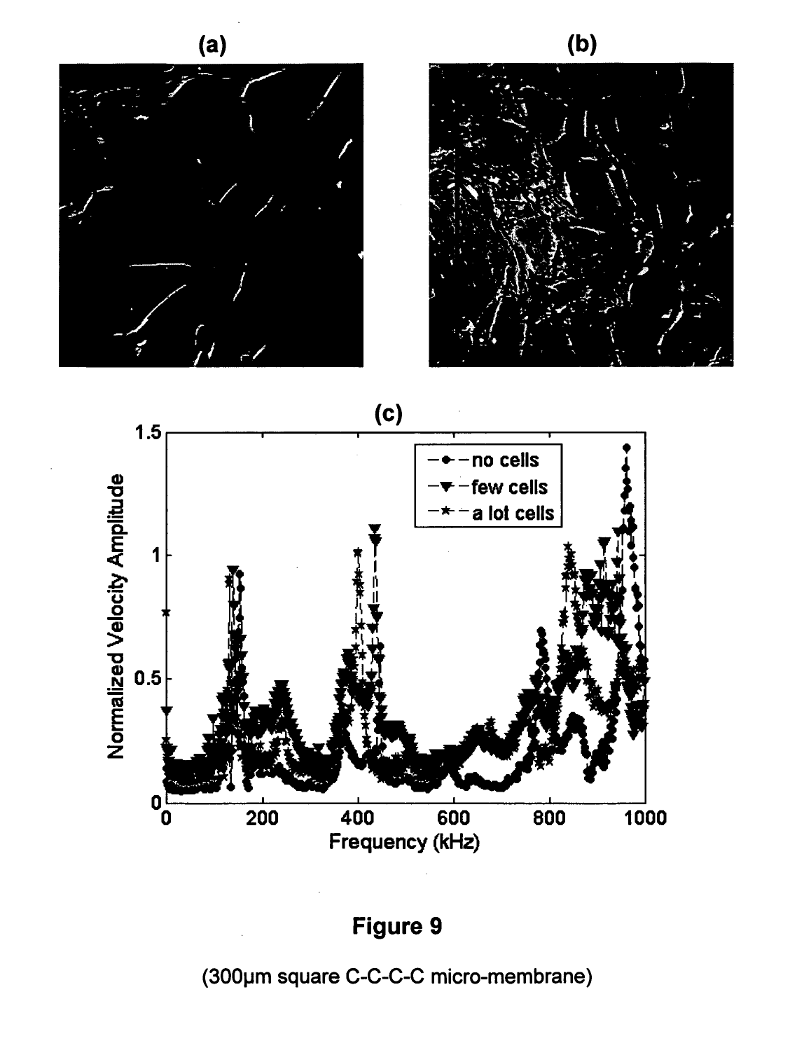

FIGS. 7, 8 and 9 illustrate the frequency response functions (FRFs) of three different types of micromembranes under three different cell densities. FIG. 7 uses a 100 μm square C-F-F-F micro-membrane; FIG. 8 uses a 200 μm square C-F-C-F micro-membrane; and FIG. 9 uses a 300 μm square C-C-C-C micro-membrane. In each case, (a) and (b) show endothelial cells coating on the surface of the micro-membrane, and (c) shows normalised velocity amplitude according to cell density.

The most dominant change of the dynamics of membrane induced by cell-loading is the shift of resonance frequencies fₙ. The first mode shapes remain almost constant, and the amplitudes of each FRF were self-normalized with respect to the amplitude of first resonant mode. Relative amplitudes of resonant modes are found to be significantly changed after the cell loading. This means that additional mass loading of attached cells on the surface of the membrane also results in the distortion of vibration shapes. The mass m or quantity of target cells can be estimated through the detection of the shift of resonance frequencies Δfₙ. Equation (1) demonstrates the relationship between mass change Δm and frequency shift Δf of a dynamic system, under the assumption that the stiffness k remains constant. This approach has been widely used in the microcantilever based biosensors.

$$f = \frac{1}{2 π} \sqrt{\frac{k}{m}} , \\ \frac{Δ m}{m} = \frac{k}{4 {π}^{2}} ( \frac{1}{{f}_{1}^{2}} - \frac{1}{{f}^{2}} ) \approx 2 \frac{Δ f}{f} \qquad ( 1 )$$

Comparing the changes of FRFs presented in FIGS. 7, 8 and 9, it is concluded that different types (dimension and boundary conditions) of the rectangular silicon micro-membranes reflect very different biosensing performance. It implies that the first type membrane (100 μm square C-F-F-F) has highest sensitivity among those three membranes, in terms of resonance frequency shift Δfₙ. It is also noted that nonlinearity occurs on the dynamics of fluid-loaded micro-membranes. In general, these experimental results demonstrate the great potential ability of micro-membrane in biosensing, even when they are immersed in a high-damping liquid environment.

The two resonant frequency based indices of Equation (2) are utilized to perform a preliminary analysis on the experimental results. FDRₙ, (Frequency Difference Ratio) is evaluated as the normalized resonant frequency difference between the cell-loaded and cell-free membrane at each measured resonance mode. AFDR is the average of all measured FDRₙ.

$${FDR}_{n} = \frac{Δ {f}_{n}}{{f}_{n}} , \\ AFDR = \frac{1}{N} {\sum }_{1}^{N} {FDR}_{n} \qquad ( 2 )$$

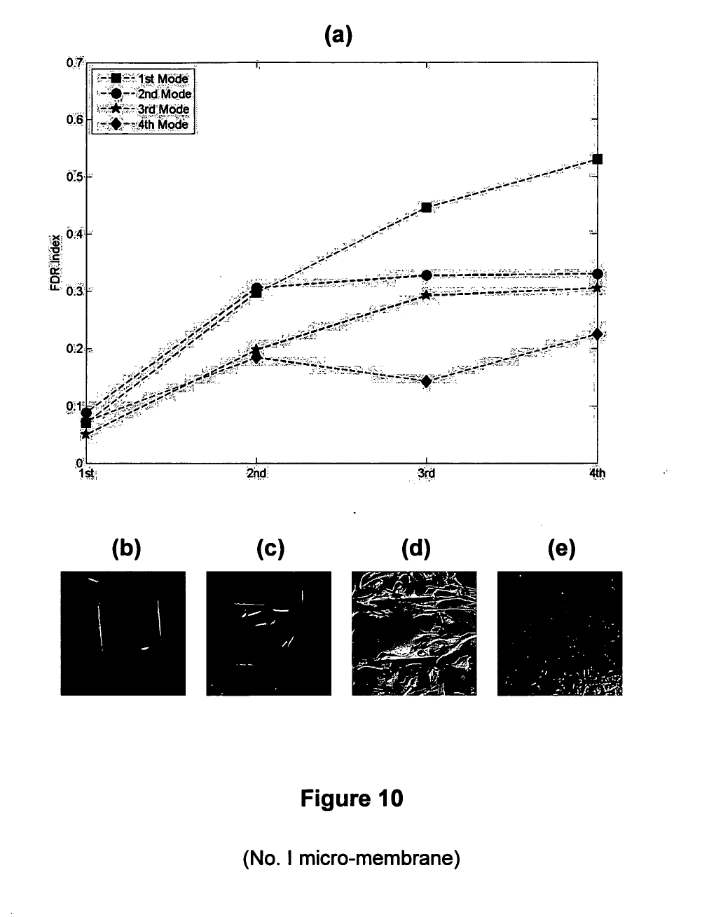

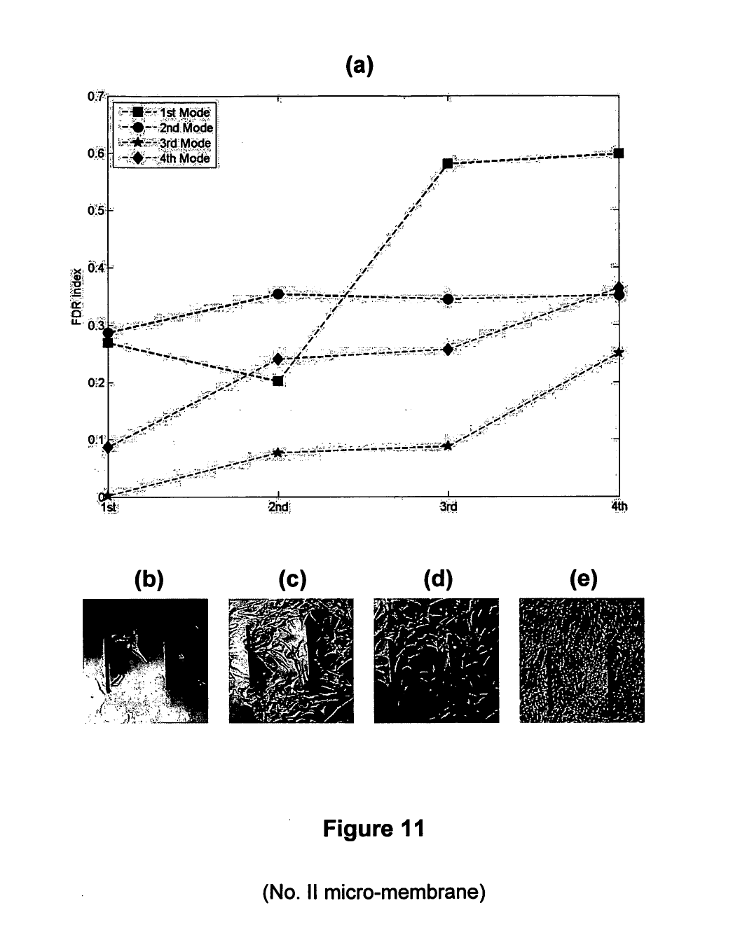

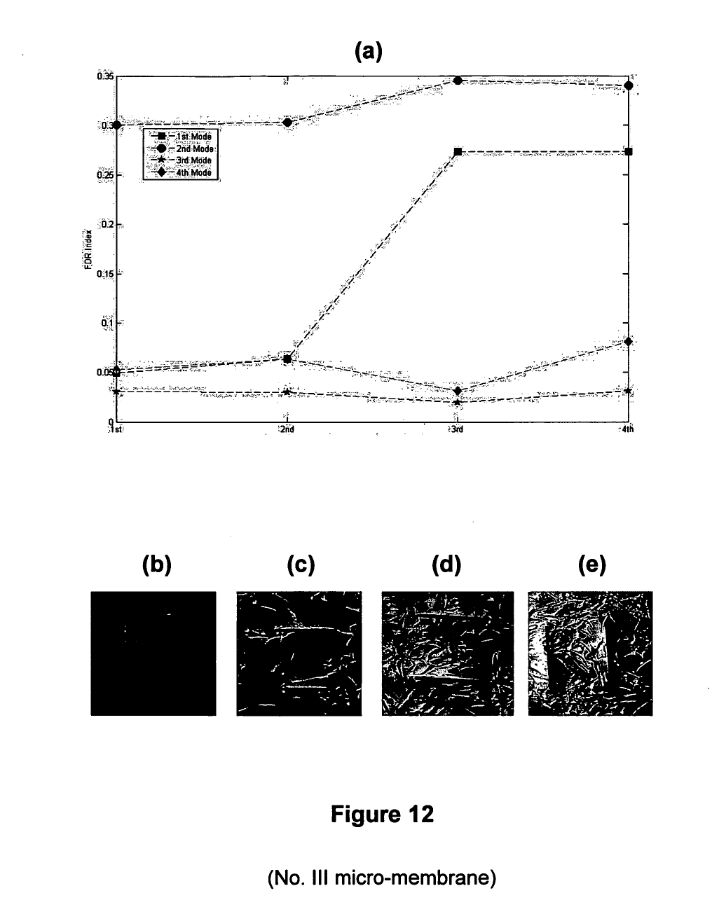

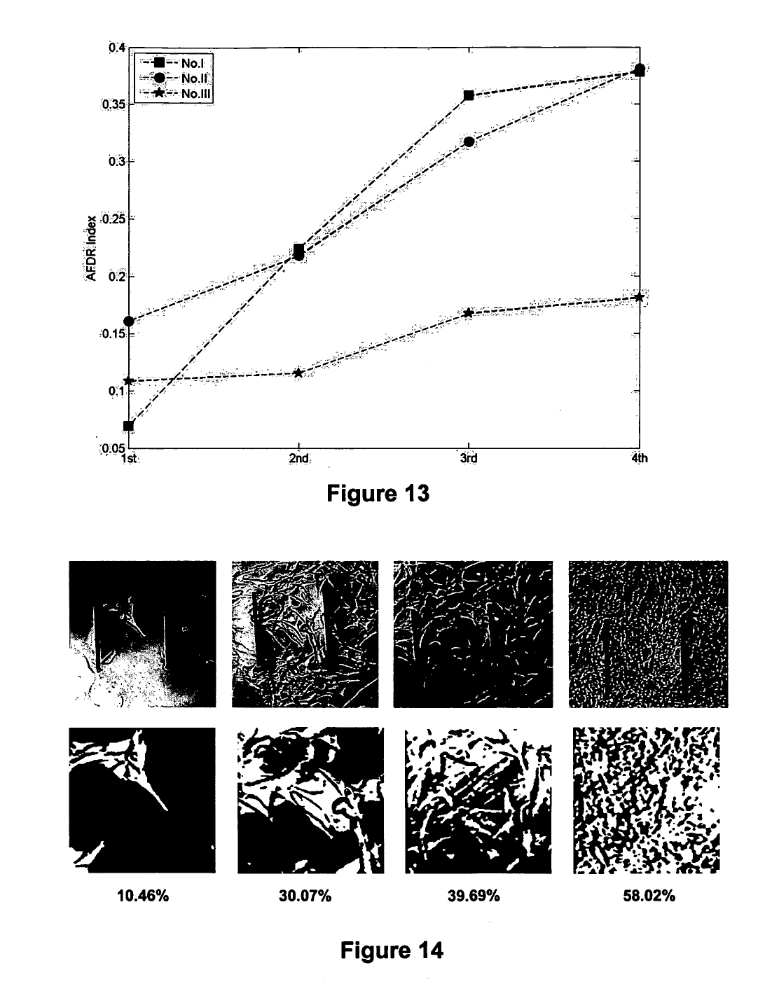

The indices of FDRₙ and AFDR evaluation were performed on three batches of bio-experimental results using three different micro-membranes, which are all approximate 200 μm square C-F-C-F membranes. The three micro-membranes are labelled as No. I, No. II and No. III respectively. In each batch of the experiment, an identical membrane was repeatedly used four times and the cell culture density was gradually increased from 25×10³/μl to 200×10³/μl. FIGS. 10, 11 and 12 illustrate the trends of the FDRₙ as the amount of cells is increased for each tested micro-membrane (No. I, No. II and No. III respectively). FIG. 13 compares the AFDR index of these three micro-membranes in each batch of experiments.

First of all, some trends of the index FDRₙ at one or two modes are not the same with the increase of cell quantity. This phenomenon is quite different with the bio-experimental results of microcantilever, where the FDR₀ of its fundamental mode always has a linearly relationship with the number of cells. The potential reasons of this phenomenon are: (a) Micro-membranes usually have much larger sensing area and carry many more cells than microcantilevers in the bio-experiments. Apart from mass change, the accumulation of cells may also result in change of structural stiffness. In such cases, the linear relationship of FDR will be violated. (b) These bio-experiments for micro-membranes are maintained in a relevant environment, for example the dynamics of microplates are measured in cell culture media. (c) Nonlinearity of the dynamics of submerged micro-membranes with randomly distributed cells exist in most experimental measurements.

On the other hand, index AFDR is capable of giving an approximate prediction of the amount of cells. The sensitivity of AFDR on these three micro-membranes is quite different. The values of AFDR for No. I and No. II membranes are very close, but that of No. III is much lower. This is due to the fact that No. I and No. II membranes were taken from the same wafer, while No. III is from a different wafer. Therefore, using the index AFDR for the micro-membrane as a biosensing platform is not a robust method. Calibration on such a biosensing device is preferable before any estimation of cell density. Considering the submerged sensing membrane as a general oscillation structure, resonant frequency fₙ can be approximately determined only by its stiffness k and mass m (see the first equation of (1)). If one assumes the system stiffness k is a constant, the mass change ratio is proportional with frequency change ratio (see the second equation of (1)). It is therefore believed that indices FDRₙ and AFDR are able to roughly reflect the cell density. However, in realistic situations, cell attachment would also affect the stiffness of sensing micro-membrane more or less, especially the endothelial cells. Hence, in some circumstances, the question is more complicated such that FDRₙ and AFDR are less useful for indicating the cell density.

### 3. Neural Network Method

On the whole, resonant frequency based indices either FDRₙ or AFDR are able to predict the cell density with only limited accuracy. This is mainly due to the complication and nonlinearities of micro-membrane sensing system. Other algorithms are desired to perform more accurate and reliable identification on cell distribution from the measured dynamics data. In this section, a simple attempt to use an artificial neural network technique to build the relationship between the sensory data and cell distribution is carried out.

In the above experimental results, LSM images were used to intuitively present the cell population in the micro-membrane sensing domain. However a quantitative index is also necessary to indicate the amount of cells for a more precise analysis. This is especially true for endothelial cells, the number of which are very hard to be count. A simple image processing procedure was carried out on each LSM image to convert it into a binary image by using the MATLAB Image Processing Toolbox. Initially the LSM image is loaded and a most clear layer is selected for the following processes, as the LSM image taken under the reflection mode usually contains three layers. Then the background image of this LSM image is created by using a morphological opening technique. Afterwards the background image is subtracted from the original image and the image contrast is enhanced, for the purpose of highlighting the area of cells occupied. Finally the corresponding binary image is created, in which the background is black and the parts of implanted cells are white. Therefore the cells population on the sensing domain can be approximately valuated by the white area ratio in this binary image. This ratio is called cell density ratio (CDR) hereafter. FIG. 14 demonstrates the results of this evaluation processes on four different LSM images, which are obtained in a same batch of bio-experiments. It can be seen that the white region of each binary image roughly indicates the shapes of endothelial cells distribution, although some local errors exist in the binary images. The evaluated ratios of white region are also listed in the bottom of FIG. 14.

However, these evaluated CDRs are not suitable to be used directly in the analysis due to the following points: (1) Apart from each cell height above the growth surface, the endothelial cells also generate a thin film over all of the culture surface. Therefore, each evaluated CDR is raised up 10% to 15% to take into account this thin film loading effect, for distinguishing from the case of no cells loading; (2) For the case that cells covered nearly the whole sensing domain (i.e. the 4th pair of images in FIG. 14), the predicted value of CDR is usually much lower than the actual situation. Therefore the predicted value needs to be increased. The modified CDRs for each experimental sample are then used as the target values in neural network applications.

Let us now consider FRF data normalization and order-reduction. Although all of the experimental settings are the same in each dynamic experiment, the amplitudes of the FRF measurements vary with experimental environment and external disturbances. Consequently it is better to normalize the measured FRFs and scale them into a same level for comparison and analysis. On the other hand, there are multiple FRF datasets in each dynamics measurement and each FRF dataset contains a very large number of frequency spectral lines. In this work, frequency spectral lines are set to be 6400 for each FRF and four sensory FRFs were recorded. Such FRF datasets are too large to directly apply into the neural network. Therefore the dimension of each FRF is reduced before the application of neural network.

For the FRF normalization, each spectrum is normalized with respect to the amplitude of its own first resonant mode. The reason for choosing the first resonant mode as the reference is based on theoretical analysis results which prove that the mass loading has the slightest effects on the first resonant mode of a rectangular membrane.

For the dimensionality reduction, the Karhunen-Loeve (K-L) decomposition method is used to extract the principal components on a multiple-FRFs dataset. The K-L decomposition is a useful method to create low dimensional, reduced-order models of dynamical systems. Assuming there are M of FRFs with N frequency in each of dynamics measurement of membrane, then this dataset forms a M×N matrix [H(ω)]_{M×N}. The process of principal components extraction of the matrix [H(ω)] using the K-L method has the following steps:
1. Firstly, a correlation matrix [C]_{M×M} is created based on the FRF matrix [H(ω)]_{M×N}.

[C]_{M×M}=[H(ω)]_{M×N}[H(ω)]_{M×N}^{T} (3)
2. The principal components are then obtained from calculating the eigenvalues and corresponding eigenvectors of matrix [C].

[C][X]=λ[X] (3)
3. Finally, the M extracted eigenvalues are examined and the first few largest eigenvalues are picked out. The eigenvectors associated with these largest eigenvalues are then considered to be the principal components and be able to represent the most significant information of the original FRF dataset.

Let us now consider dataset creation. The dynamics (FRF) of 4 different used membranes without any cells loading are also provided in the dataset as references. Two additional samples are also provided for the purpose of validation. Consequently there are 18 different samples in total created for training and validation of the neural network. The eigenvectors related to the largest eigenvalue of FRF dataset of each sample are extracted as the neural network input and the CDRs of every samples are calculated as the neural network targets.

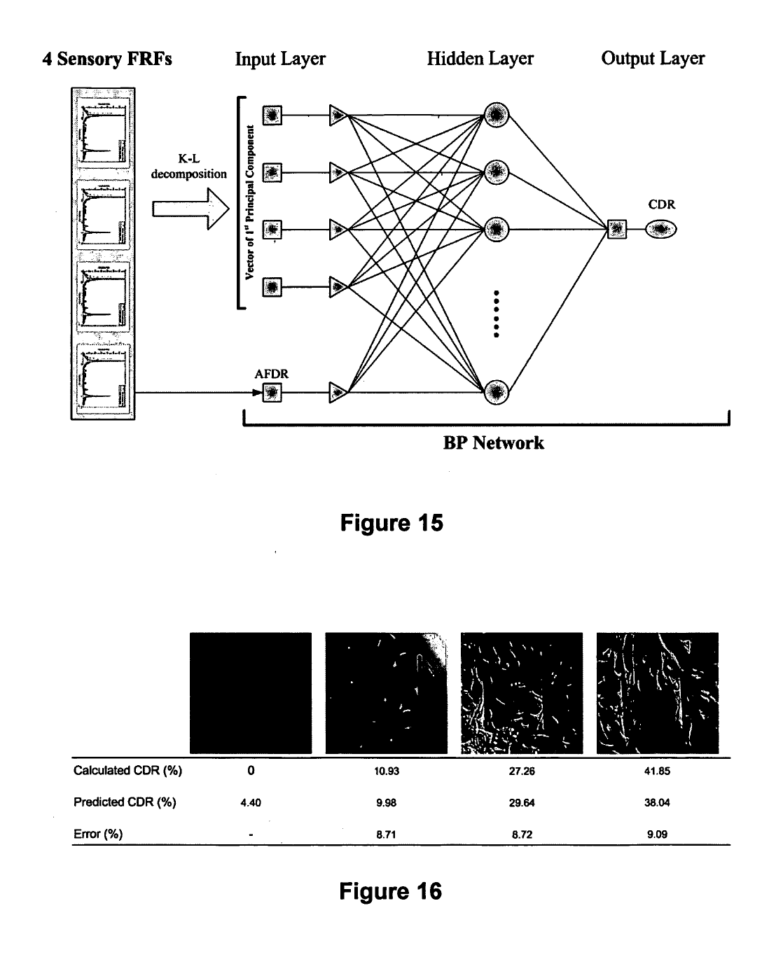

Let us now consider network design and training. The widely used back-propagation (BP) neural network was selected to predict cells density. FIG. 15 illustrates the concept of using BP neural network to predict the value of CDR. Besides the principal components extracted from FRF datasets, the value of index AFDR of each sample provide an additional input to the neural network. As the index of AFDR proved to be highly related to cells distribution in the last section, it then can help the neural network to achieve a fast convergence and good predictions. Among the 18 samples in the dataset, the first 14 samples are used for training neural network and the left 4 samples are used for validation. As the number of samples are limited, it is more sensible to design and use a simple neural network rather than a complicated one. The BP neural network used here is designed to have only one hidden layer with few neurons. Several trails with different number of hidden layer neurons were carried out to test the differences on the normalized system error. The hidden layer with 5 neurons produces the best performance. The training process of BP network herein establishes an approximate function (nonlinear regression) between the inputs and targets, through iteratively adjusting the weights and biases of network to meet a setting goal (mean square error). The training parameters can affect the network convergence speed as well as the final predication accuracy. Bad parameters may lead to very slow training processes or over-fitting results. Several tests were then carried out to find out reasonable training parameters. The final training parameters used here are selected as: moment rate is 0.9, learning rate is 0.1, the maximum error is 0.001 and the maximum number of iterations is 3000.

FIG. 16 demonstrates the prediction results of CDR on sample numbers 15 to 18 obtained from the training result of BP network. The prediction results are very well matched with the CDR values calculated from corresponding LSM images.

Although preferred embodiments of the invention have been described, it is to be understood that these are by way of example only and that various modifications may be contemplated.

1. A method of characterising a property or behaviour of at least one biological cell, the method comprising the steps of:
providing a microplate;
submerging at least one surface of the microplate in a cell culture medium such that at least one biological cell to be characterised is in contact with the microplate;
vibrating the microplate;
providing a plurality of mutually spaced sensors coupled to the microplate;
obtaining a respective sensory data time series from each sensor during vibration of the microplate, the microplate and the sensors being arranged such that the obtained sensory data time series are not independent from one another;
processing the sensory data time series so as to characterise a property or behaviour of the at least one biological cell.

2. The method of claim 1 wherein the processing step comprises:
specifying a cell dynamic behaviour category; and
processing the sensory data time series so as to determine whether the dynamic behaviour of the at least one cell is in the specified cell dynamic behaviour category.

3. The method of claim 1 wherein the processing step comprises:
specifying a cell property; and
processing the sensory data time series so as to determine a measurement of the specified property of the at least one cell.

4. The method of claim 1 wherein the processing step comprises analysing the sensory data time series in one or more of the time domain, the frequency domain and the wavelet domain.

5. The method of claim 1 wherein the processing step comprises analysing frequency response functions (FRFs).

6. The method of claim 1 wherein the processing step comprises using one or more of a neural network and Karhunen-Loeve decomposition.

7. The method of claim 1 wherein the step of vibrating the microplate comprising vibrating the microplate periodically.

8. The method of claim 1 wherein the step of vibrating the microplate comprising vibrating the microplate randomly.

9. A system for characterising a property or behaviour of at least one biological cell, the system comprising:
a container for cell culture medium;
a microplate disposed within the container such that, when the container is at least partially filled with cell culture medium, at least one surface of the microplate is submerged in the cell culture medium;
at least one actuator operable to vibrate the microplate;
a plurality of mutually spaced sensors coupled to the microplate, each sensor being operable to provide a respective sensory data time series during vibration of the microplate, the microplate and the sensors being arranged such that the provided sensory data time series are not independent from one another; and
a processor operable to receive the sensory data time series from the sensors and to process the received sensory data time series so as to characterise a property or behaviour of at least one biological cell in contact with the microplate.

10. The system of claim 9 wherein the microplate boundary conditions are selected from clamped, cantilever, free and point-supported.

11. The system of claim 9 wherein the sensors are selected from piezoresistive gauge sensors, optical sensors, strain sensors and acceleration sensors.

12. The system of 9 wherein the at least one actuator comprises a piezoelectric transducer.

13. The system of claim 9 wherein the at least one actuator comprises a sonic actuator.

14. The system of claim 9 wherein the container is a Petri-dish.
